(19) United States
(12) Patent Application Publication
(10) Pub. No.: US 2021/0237040 A1
(43) Pub. Date: Aug. 5, 2021

(54) METAL-IODIDE CATALYTIC SYSTEM FOR DIRECT ETHERIFICATION FROM ALDEHYDES AND/OR KETONES

(71) Applicants: RHODIA OPERATIONS, Aubervilliers (FR); LE CENTRE NATIONAL DE LA RECHERCHE SCIENTIFIQUE, Paris Cedex 16 (FR); UNIVERSITE DE LILE, Lille (FR); CENTRALE LILLE INSTITUT, Villeneuve d'Ascq Cedex (FR)

(72) Inventors: Dan WU, Villeneuve d'Ascq (FR); Vitaly ORDOMSKY, Villeneuve d'Ascq (FR); Willinton Yesid HERNANDEZ ENCISO, Shanghai (CN); Stéphane STREIFF, Shanghai (CN)

(21) Appl. No.: 15/734,573

(22) PCT Filed: Jun. 26, 2018
(86) PCT No.: PCT/CN2018/092796

§ 371 Date: Dec. 3, 2020

(51) Int. Cl. B01J 27/08; B01J 23/42; B01J 21/04; B01J 23/46; B01J 23/44; B01J 21/18; B01J 37/22; C07D 307/42; C07C 41/01

(52) U.S. Cl. CPC B01J 27/08; B01J 23/42; B01J 21/04; B01J 23/462; C07C 41/01; B01J 21/18; B01J 37/22; C07D 307/42; B01J 23/44

(57) ABSTRACT

A process for etherification of aldehydes and/or ketones in the presence of a catalyst and an iodine source. In particular, a process for the synthesis of an ether compound, comprising reacting an aldehyde and/or a ketone with an alcohol, in the presence of (i) a metal/support heterogeneous catalyst and an iodine source, or (ii) a metal-iodine catalyst, in a reactor, whereby the ether compound is obtained. A catalytic system comprising a metal/support heterogeneous catalyst and an iodine source, and a process for its preparation.

### FIELD OF THE INVENTION

The present invention relates to a process for preparing an ether compound by reacting an aldehyde and/or a ketone with an alcohol in the presence of a metal-iodine catalyst or a metal catalyst with an iodine source.

### BACKGROUND

Ethers are widely used in synthetic organic chemistry for example as solvents or starting materials for further synthesis applications. Several routes to prepare ethers are well known, including dehydration of alcohols, Williamson ether synthesis, Wurtz's synthesis, Ullmann condensation or the electrophilic addition of alcohols to alkenes.

Generally, it is a common problem, that in the conventionally used etherification reactions the required temperatures are costly and lead to low product yields. It is desired to establish eco-friendly lower reaction temperatures and thereby obtain higher product yields at lower production costs. Therefore, there is intense need to not only improve the current syntheses approaches but also search for new etherification routes applicable in industrial scale.

Recently, Doyle et al. (J. Am. Chem. Soc. 1972, 94, 3659) have reported on an acid promoted reductive coupling of carbonyl compounds with trialkylsilanes as a route to symmetrical ethers. This method is limited in its large-scale use by its necessity of several-fold excess of strong Bronsted acids. Also, Yadav et al. (Tetrahedron Lett. 2010, 51, 46) have reported on the reductive etherification of carbonyl compounds using polymethylhydrosiloxane in the presence of a catalytic amount of molecular iodine to afford the corresponding symmetrical ethers.

For product diversity it is desired to obtain not only symmetrical ethers but to chemoselectively react two different starting materials to obtain unsymmetrical ethers. In this context, recently, the reductive condensation of alcohols and aldehydes or ketones with a Zr-based catalyst to generate ethers has been reported by Suhas et al. (ChemSusChem 2017, 10, 4090) as an innovative route to valorize different building block units sourced from biomass. Also, Seidel et al (J. Am. Chem. Soc. 2017, 139, 10224), Albrecht et al. (Catal. Sci. Technol. 2017, 7, 5766) and Bell et al. (ChemSusChem 2017, 10, 2527) reported on methods for catalyzed etherification of aldehydes or ketones. The catalytic systems reported for these etherification reactions are normally composed by the combination of an homogeneous or heterogeneous hydrogenation catalyst (e.g. Zr-based catalysts, iridium(III) Cp* complex or Pd/C, respectively), a Bronsted acid catalyst (e.g. 4-ethylbenzenesulfonic acid, HCl, Amberlyst 15) and in some cases, an alkyl/aryl silane as reducing agent (e.g. Et₃SiH, PhSiH). These catalytic systems have shown to be very effective on a very broad substrate scope, affording high selectivity to the targeted ethers (over 90%). On the other hand, regardless the homogeneous or heterogeneous nature of the catalyst, the necessity of a multicomponent catalytic system can be seen as a serious drawback for the implementation of this process in an industrial scale.

Qinglei et al. (Nat. Commun. 2017, 1) have reported on bromide salt-modified Pd/C in H₂O/CH₂Cl₂ to efficiently catalyze the transformation of aromatic ethers, which can be derived from biomass, to cyclohexanone and its derivatives via hydrogenation and hydrolysis processes. The modification of Pd-supported catalysts with halides (e.g. Pd/C or Pd/Al₂O₃ catalysts modified with Br) has been presented by Choudhary et al. (Chem. Commun. 2004, 2054) as a way to fine tune the hydrogenation properties of palladium in different reactions, such as H₂O₂ synthesis and production of ketones from aromatic ethers. The presence of the halide in close interaction with Pd cause delocalization of the electronic density on the metal surface, which can affect either the selective interaction of palladium with the molecules in the reaction medium, and/or the stabilization of different reaction intermediates.

The inventors of the present invention have surprisingly found that this type of Pd-halogen interaction can be implemented for the selective synthesis of ethers via reductive coupling of alcohols with aldehydes or ketones and also enhanced this technique to other catalytically active precious metals like Ru, Rh, Ir, Pt.

### SUMMARY OF THE INVENTION

The problem to be solved by the present invention is to provide a process for the etherification of aldehydes and/or ketones with alcohols at lower temperatures than those that are currently used. Due to the milder reaction conditions higher product yields are obtained. Furthermore, due to the lower temperature the process provided by the present invention is cheaper and environmentally friendly. The starting materials used for the process of the present invention may be obtained for example from biomass-derived molecules.

Accordingly, the present invention provides a catalytic system comprising a precious metal and an iodine source for reductive condensation of aldehydes and/or ketones with alcohols to produce ethers at very mild reaction conditions. The catalytic system can be reused several times without losing its catalytic efficiency. Even more, the catalytic system remains active after washing, corroborating the heterogeneous nature of the catalytic system.

The catalytic system of the present invention is using an easy-to-produce heterogeneous catalyst and is effective on a very broad substrates scope. Therefore, the present invention provides a very elegant and versatile way to produce ethers and polyethers.

The invention therefore relates to the subject-matter defined in the following items 1 to 50:

1. A process for the synthesis of an ether compound, comprising reacting

an aldehyde and/or a ketone with an alcohol, in the presence of (i) a metal/support heterogeneous catalyst and an iodine source or (ii) a metal-iodine catalyst,
whereby the ether compound is obtained.

2. The process according to item 1, wherein the ether compound is a polyether.

3. The process according to item 1 or 2, wherein the ether compound is selected from the group consisting of dioctyl ethers, benzyl ethers, 2-(alkoxymethyl)furans and polyethers from 2,5-diformyl furan.

4. The process according to any of items 1-3, further comprising providing a protected aldehyde and/or a protected ketone, and deprotecting the protected aldehyde and/or the protected ketone to obtain said aldehyde and/or said ketone prior to said reacting step.

5. The process according to any one of items 1 to 4, further comprising providing a protected alcohol, and deprotecting the protected alcohol to obtain said alcohol prior to said reacting step.

6. The process according to any of items 1-5, wherein the aldehyde is selected from the group consisting of monoaldehydes and dialdehydes; and/or wherein the aldehyde is selected from the group consisting of 1-Octanal, 1-Heptanal, Benzyl aldehyde, Furfural and Vanillin.

7. The process according to any of items 1-6, wherein the alcohol is a monovalent alcohol.

8. The process according to any of items 1-7, wherein the alcohol is selected from the group consisting of octanol, isopropanol, benzyl alcohol, methanol, isopropanol, tert-amyl alcohol and furfuryl alcohol.

9. The process according to any of items 1-8, wherein the iodine source is an organic iodide or I₂.

10. The process according to item 1-9, wherein the iodine source is selected from the group consisting of alkyl iodides, alkenyl iodides, alkynyl iodides, aryl iodides, CH₃CH₂I, HI and I₂.

11. The process according to any of items 1-10, wherein the metal/support heterogenic catalyst comprises a support and a catalytically active metal.

12. The process according to item 11, wherein the catalytically active metal is Ru.

13. The process according to item 11, wherein the catalytically active metal is Rh.

14. The process according to item 11, wherein the catalytically active metal is Ir.

15. The process according to item 11, wherein the catalytically active metal is Pd.

16. The process according to item 11, wherein the catalytically active metal is Pt.

17. The process according to any of items 1-11, wherein the catalyst and the iodine source are a bifunctional Pd-iodide catalytic system.

18. The process according to any of items 1-17, wherein the metal/support heterogeneous catalyst is Pd/Al₂O₃, and wherein the iodine source is CH₃CH₂.

19. The process according to any of items 1-18, wherein the process is carried out at a temperature in the range from 50 to 90° C.

20. The process according to any of items 1-19 wherein the process is carried out at a temperature in the range from 55-80° C.

21. The process according to any of items 1-20, wherein the process is carried out at a temperature in the range from 56-65° C.

22. The process according to any of items 1-21, wherein the process is carried out at a temperature in the range from 57-63° C.

23. The process according to any of items 1-22, wherein the process is carried out at a temperature in the range from 56-65° C.

24. The process according to any of items 1-23, wherein the process is carried out at a temperature in the range from 57-63° C.

25. The process according to any of items 1-24, wherein the process is carried out at a temperature in the range from 58-62° C.

26. The process according to any of items 1-25, wherein the process is carried out at a temperature in the range from 59-61° C.

27. The process according to any of items 1-26, wherein the process is carried out at a temperature of about 60° C.

28. The process according to any of items 1-27, wherein the process is carried out in a reactor.

29. The process according to any of items 1-28, wherein the reactor is a stainless steel reactor.

30. The process according to any of items 1-29, wherein the process is performed at an H₂-pressure in the range from 10-30 bar.

31. The process according to any of items 1-30, wherein the process is performed at an H₂-pressure in the range from 15-25 bar.

32. The process according to any of items 1-31, wherein the process is performed at an H₂-pressure in the range from 17-23 bar.

33. The process according to any of items 1-32, wherein the process is performed at an H₂-pressure in the range from 18-22 bar.

34. The process according to any of items 1-33, wherein the process is performed at an H₂-pressure in the range from 19-21 bar.

35. The process according to any of items 1-34, wherein the process is performed at an H₂-pressure of 20 bar.

36. The process according to any of items 1-35, wherein the reacting is performed for a time period in the range from 1-20 h.

37. The process according to any of items 1-35, wherein the reacting is performed for a time period in the range from 2-15 h.

38. The process according to any of items 1-35, wherein the reacting is performed for about 2 h.

39. The process according to any of items 1-35, wherein the reacting is performed for about 3 h.

40. The process according to any of items 1-35, wherein the reacting is performed for about 6 h.

41. The process according to any of items 1-35, wherein the reacting is performed for about 15 h.

42. The process according to any of items 1-41, wherein the reaction is carried out under continuous stirring.

43. A catalytic system comprising a metal/support heterogeneous catalyst, and an iodine source, wherein the metal is selected from the group consisting of Ru, Rh, Ir, Pd and Pt, and the support is selected from the group consisting of Al₂O₃, ZrO₂, C, SiO₂ and Ga₂O₃, and wherein (i) the iodine source is selected from the group consisting of organic iodides, HI and I₂, and/or (ii) said catalytic system has an H₂-pressure in the range from 10-30 bar, preferably from 15-25 bar, more preferably from 17-23 bar, even more preferably from 18-22 bar, yet even more preferably from 19-21 bar, most preferably of about 20 bar.

44. The catalytic system according to item 43, wherein the organic iodide is selected from the group consisting of alkyl iodides, alkenyl iodides, alkynyl iodides and aryl iodides.

45. The catalytic system according to item 43 or 44, wherein the organic iodide is selected from the group consisting of methyl iodide, ethyl iodide, propyl iodide, butyl iodide, pentyl iodide, hexyl iodide, heptyl iodide, octyl iodide, nonyl iodide and decyl iodide.

46. The catalytic system according to item 43 or 44, wherein the organic iodide is selected from the group consisting of ethenyl iodide, propenyl iodide, butenyl iodide, pentenyl iodide, hexenyl iodide, heptenyl iodide, octenyl iodide, nonenyl iodide and decenyl iodide.

47. The catalytic system according to item 43 or 44, wherein the organic iodide is selected from the group consisting of ethynyl iodide, propynyl iodide, butynyl iodide, pentynyl iodide, hexynyl iodide, heptynyl iodide, octynyl iodide, nonynyl iodide and decynyl iodide.

48. The catalytic system according to any of items 43-47, wherein the metal/support heterogeneous catalyst is Pd/Al₂O₃.

49. The catalytic system according to any of items 43-48, which is a bifunctional catalytic system.

50. A process for preparation of a catalytic system, wherein said process comprises contacting a metal/support heterogeneous catalyst and an iodine source selected from the group consisting of organic iodides, HI and I₂ under an H₂-pressure in the range from 10-30 bar, preferably from 15-25 bar, more preferably from 17-23 bar, even more preferably from 18-22 bar, yet even more preferably from 19-21 bar, most preferably of about 20 bar.

### DETAILED DESCRIPTION

The present invention relates to a chemoselective Pd—I catalytic system for reductive condensation of an aldehyde and/or ketone with an alcohol to produce ethers at very mild reaction conditions.

The method of the invention comprises reacting an aldehyde and/or a ketone with an alcohol. In one embodiment of the present invention the method comprises reacting an aldehyde with an alcohol. In another embodiment of the present invention the method comprises reacting a ketone with an alcohol. In yet another embodiment of the present invention the method comprises reacting an aldehyde and a ketone with an alcohol. The term aldehyde refers to organic compounds comprising at least one aldehyde group, and the term ketone refers to organic compounds comprising at least one keto group. The term aldehyde includes a monoaldehyde as well as a dialdehyde. Specific examples for aldehydes include, but are not limited to, methanal, 1-ethanal, 1-propanal, 1-butanal, 1-pentanal, 1-hexanal, 1-heptanal, 1-octanal, 1-nonanal, 1-decanal, oxaldehyde, propanedial butanedial, pentanedial, hexanedial, heptanedial, ocatanedial, nonanedial, decanedial, benzyl aldehyde, furfural and vanillin. Preferred aldehydes are 1-heptanal, 1-octanal, benzyl aldehyde, furfural and vanillin. Aldehydes might contain two or more carbonyl groups like 2,5-diformylfuran which can all react to form ether groups.

The carbonyl group of the aldehyde or ketone, and/or the hydroxyl group of the alcohol may be, independently from each other, protected by a suitable protecting group. Protecting groups are well known to those skilled in the art. The protecting group is typically removed in the process prior to reacting the aldehyde and/or ketone with the alcohol by treatment with an adequate protecting group removing agent in an adequate amount. Protecting group removing agents are well known to those skilled in the art. The choice of the protecting group removing agent depends on the selected protecting group.

In one embodiment the method of the invention further comprises providing a protected aldehyde and/or a protected ketone, and removing the protecting group(s) by contacting the protected aldehyde and/or the protected ketone with a suitable protecting group removing agent, to obtain the aldehyde and/or ketone. In an alternative embodiment the method of the invention further comprises adding a protected aldehyde and/or a protected ketone to the reaction mixture, and removing the protecting group(s) by contacting the protected aldehyde and/or the protected ketone with a suitable protecting group removing agent, to obtain the aldehyde and/or ketone.

In another embodiment the method of the invention further comprises providing a protected alcohol, and removing the protecting group(s) by contacting the protecting group(s) by contacting the protected alcohol with a suitable protecting group removing agent, to obtain the alcohol. In an alternative embodiment the method of the invention further comprises adding a protected alcohol to the reaction mixture, and removing the protecting group(s) by contacting the protected alcohol with a suitable protecting group removing agent, to obtain the alcohol.

Generally, carbonyl groups can be protected by carbonyl protecting groups. Carbonyl protecting groups include, for example, acetals, ketals, acylals and dithianes.

Generally, hydroxyl groups can be protected by hydroxyl protecting groups. Hydroxyl protecting groups include, for example, acetyl (Ac), benzoyl (Bz), benzyl (Bn), β-methoxyethoxymethyl ether (MEM), methoxymethyl ether (MOM), methoxytrityl [(4-methoxyphenyl)diphenylmethyl] (MMT), p-methoxybenzyl ether (PMB), methylthiomethyl ether, Pivaloyl (Piv), Tetrahydropyranyl (THP), Tetrahydrofuran (THF), Trityl (triphenylmethyl, Tr), trimethylsilyl (TMS), tert-butyldimethylsilyl (TBDMS), tri-iso-propylsilyloxymethyl (TOM), and triisopropylsilyl (TIPS), methyl ethers, ethoxyethyl ethers (EE).

Acetals and ketals can be removed by acid. Acylals can be removed by Lewis acids. Dithianes can be removed by metal salts or oxidizing agents.

Acetyl (Ac) can be removed by acid or base. Benzoyl (Bz) can be removed by acid or base. Benzyl (Bn) can be removed by hydrogenolysis. B-Methoxyethoxymethyl ether (MEM) can be removed by acid. Dimethoxytrityl, [bis-(4-methoxyphenyl)phenylmethyl] (DMT) can be removed by weak acid. Methoxymethyl ether (MOM) can be removed by acid. Methoxytrityl [(4-methoxyphenyl)diphenylmethyl] (MMT) can be removed by acid and hydrogenolysis. p-Methoxybenzyl ether (PMB) can be removed by acid, hydrogenolysis, or oxidation. Methylthiomethyl ether can be removed by acid. Pivaloyl (Piv) can be removed by acid, base or reductant agents. Tetrahydropyranyl (THP) can be removed by acid. Tetrahydrofuran (THF) can be removed by acid. Trityl (triphenylmethyl, Tr) can be removed by acid and hydrogenolysis. Trimethylsilyl (TMS), tert-butyldimethylsilyl (TBDMS), tri-iso-propylsilyloxymethyl (TOM), and triisopropylsilyl (TIPS) can be removed by acid or fluoride ion, such as NaF, TBAF (tetra-n-butylammonium fluoride), HF-Py, or HF-NEt3. Methyl ethers can be cleaved by TMSI in dichloromethane or acetonitrile or chloroform or by BBr₃ in DCM. Ethoxyethyl ethers (EE) can be cleaved e.g. by iN hydrochloric acid.

Protecting groups and their application are described in detail in Peter G. M. Wuts, “Greene's Protective Groups in Organic Synthesis”, 5^{th} ed., 2014 (ISBN 9781118905128).

In accordance with the invention the aldehyde and/or ketone reacts with an alcohol. The term alcohol comprises alkanols. Specific examples of alcohols according to the invention include, but are not limited to, methanol, ethanol, 1-propanol, 1-butanol, 1-pentanol, 1-hexanol, 1-heptanol, 1-octanol, 1-nonanol, 1-decanol, isopropanol, benzyl alcohol, tert-amyl alcohol and furfuryl alcohol. preferred alcohols are 1-ocatanol, isopropanol, benzyl alcohol, tert-amyl alcohol and furfuryl alcohol. Alcohol might contain two or more hydroxyl groups like ethylene glycol which all react to form ether groups.

In a specific embodiment a single compound can be both (i) the aldehyde or ketone and (ii) the alcohol. Such compounds comprise a at least one aldehyde or keto group and at least one hydroxyl group. An example for such a compound is 5-(hydroxymethyl)furfural.

Ether compounds that can be produced by the process according to the invention comprise, but are not limited to 1-(octyloxy)heptane, 1-(octyloxy)octane, 1-(octyloxy)nonane, (isopropoxymethyl)benzene, (oxybis(methylene))dibenzene, 2-(methoxymethyl)furan, 2-(isopropoxymethyl)furan, 2-((tert-pentyloxy)methyl)furan, 2-((octyl)methyl)furan, 2,2′-(oxybis(methylene))difuran, dioctyl ethers, benzyl ethers, 2-(alkoxymethyl)furans and polyethers from 2,5-diformyl furan.

The method of the present invention allows the production of polyether compounds in the case if polyaldehydes and polyalcohols are used or molecules containing both carbonyl and hydroxyl groups (like 5-(hydroxymethyl)furfural, e.g. according to the following schemes:

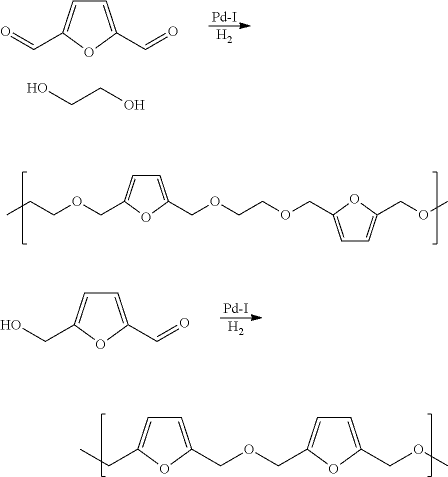

According to the invention the catalyst is a metal/support heterogeneous catalyst. The term “metal/support catalyst” refers to a catalyst comprising a catalytically active metal and a support. The phase of a “heterogeneous” catalyst differs from that of the reactants. The heterogeneous catalyst preferably is a solid catalyst. The metal/support catalyst preferably is a catalyst comprising a catalytically active metal and a solid support. Catalytically active metals according to the invention are metals selected from the group consisting of precious metals, preferably Ru, Rh, Ir, Pd and/or Pt. Pd is most preferred. Supports according to the invention can be porous inorganic compounds like Al₂O₃, SiO₂, ZrO₂ or Ga₂O₃. In a preferred embodiment the support is Al₂O₃. In a preferred embodiment the metal/support heterogeneous catalyst is Pd/Al₂O₃.

According to the invention the iodine source can be an iodide. Generally, any compound that is able to provide iodide or iodine (I) in its ionic or elemental form is suitable as iodine source. Some possible iodine sources according to the invention comprise, but are not limited to, alkyl iodides, alkenyl iodides, alkynyl iodides aryl iodides, HI and I₂. Preferred iodine sources are organic iodides. Organic iodides are organic compounds containing a carbon-iodine (C—I) bond. In organic iodides the carbon-iodine bond is weaker than corresponding carbon-halogen bonds of other organic halogenides due to the poor electronegative nature of the iodine atom. Organic iodides can be any linear or branched alkyl iodide, linear or branched alkenyl iodide, or linear or branched alkynyl iodide.

Alkyl iodides according to the invention have the general formula CₙH₂ₙ₊₁I.

Therefore, alkyl iodides according to the invention comprise methyl iodide (CH₃I), ethyl iodide (CH₃CH₂I), propyl iodide (CH₃CH₂CH₂I), butyl iodide (CH₃(CH₂)₂CH₂I), pentyl iodide (CH₃(CH₂)₃CH₂I), hexyl iodide (CH₃(CH₂)₄CH₂I), heptyl iodide (CH₃(CH₂)₅CH₂I), octyl iodide (CH₃(CH₂)₆CH₂I), nonyl iodide (CH₃(CH₂)₇CH₂I) and decyl iodide (CH₃(CH₂)₈CH₂I).

Alkenyl iodides according to the invention have the general formula CₙH₂ₙ₋₁I. Preferably, alkenyl iodides according to the invention comprise ethenyl iodide (CH₂CHI), propenyl iodide, butenyl iodide, pentenyl iodide, hexenyl iodide, heptenyl iodide, octenyl iodide, nonenyl iodide and decenyl iodide.

Alkenyl iodides according to the invention have the general formula CₙH₂ₙ₋₃I. Preferably alkynyl iodides according to the invention comprise ethynyl iodide (CHCI), propynyl iodide, butynyl iodide, pentynyl iodide, hexynyl iodide, heptynyl iodide, octynyl iodide, nonynyl iodide and decynyl iodide.

Especially preferred iodine sources are CH₃I, CH₃CH₂I, HI and I₂. Especially preferred is CH₃CH₂I.

According to the invention the support and the catalytically active metal (metal) form a metal/support heterogeneous catalyst (also called catalyst). The catalyst and the iodine source form a catalytic system M-I. The catalytic system M-I is formed by interaction of the metal/support catalyst with the iodine source in the reaction mixture with formation of M-I species on the surface of the metal. The catalytic system can be reused at least twice, preferably at least three times, more preferably at least four times, even more preferably at least 5 times without losing efficiency neither in conversion nor selectivity. Although the formation of the highly active and selective catalytic species usually requires the “in situ” activation of the M-I interactions under reaction conditions (first catalytic run), the catalyst can be reused several times without losing its catalytic efficiency. Even more, the catalyst remains active after washing, corroborating the heterogeneous nature of the M-I catalytic system. In a preferred embodiment the catalytic system is a Pd—I catalytic system.

In another preferred embodiment, the catalytic system is a bifunctional catalytic system. The term “bifunctional” refers to the finding that hydrogen dissociated over Pd interacts with grafted I and becomes H⁺ which appears to play the crucial role in this reaction.

The catalytic system can be directly formed in the reaction mixture by mixing the iodine source with the metal/support heterogeneous catalyst. In one embodiment of the present invention the catalytic system is obtained by mixing the Pd/Al₂O₃ heterogeneous catalyst with ethyl iodide in the reaction mixture. In another embodiment of the present invention the catalytic system of the present invention can be obtained by pretreatment of the metal/support heterogeneous catalyst with the iodine source by chemical vapor deposition or just mixing the metal/support heterogeneous catalyst with the iodine source. In yet another embodiment a Pd—I catalytic system of the present invention is obtained by chemical vapor deposition. Chemical vapor deposition is a method well known to a person skilled in the art.

With the process according to the invention it is possible to produce ethers at very mild reaction conditions at about 60° C. For instance, by using the Pd—I catalytic system described herein, the etherification reaction of furfural and isopropanol allow a higher yield of 2-(isopropoxymethyl) furan than that reported in literature (97% vs 67%, respectively) at a lower temperature (60° C. instead of 100° C.). Accordingly, the process of the present invention may be performed at a temperature in the range from 50-90° C., preferably at a temperature in the range from 55-80° C., more preferably at a temperature in the range from 56-65° C., even more preferably at a temperature in the range from 57-63° C., yet even more preferably at a temperature in the range from 58-62° C., yet even far more preferably at a temperature in the range from 59-61° C., most preferably at a temperature of about 60° C.

Typically, in the process of the present invention the reaction is performed under H₂-pressure. Preferably the H₂ pressure is in the range from 10-30 bar, preferably at an H₂-pressure in the range from 15-25 bar, more preferably at an H₂-pressure in the range from 17-23 bar, even more preferably at an H₂-pressure in the range from 18-22 bar, yet even more preferably at an H₂-pressure in the range from 19-21 bar, most preferably at an H₂-pressure of about 20 bar.

In addition, the effectiveness of this catalytic system on a very broad substrates scope (see tables 1, 2 and 3) highlights on the generated Pd—I interactions as a very elegant and versatile way to produce ethers and polyethers from biomass-derived molecules, using an easy-to-produce heterogeneous catalyst.

The process according to the invention can be used for the synthesis of ethers from mono aldehydes and/or ketones and polyethers from dialdehydes and/or ketones.

The proposed mechanism of the reaction involves use of basic side I and nearby metallic Pd site.

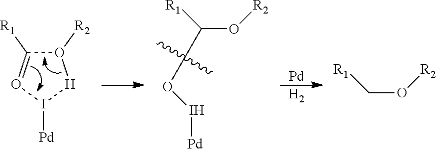

Another aspect of the present invention is a catalytic system useful for carrying out the process of the invention. Yet another aspect of the invention is a process for preparing such a catalytic system. The embodiments described above with respect to the process for the synthesis of an ether compound apply to the other aspects of the invention mutatis mutandis.

### EXAMPLES

Syntheses Experiments:

1. Catalysis Over Pd Catalyst with Ethyl Iodide in the Reaction Mixture

1.1 Etherification of Alkyl Aldehydes:

In a typical experiment, a 50 ml stainless steel reactor was filled with 2 g of 1-octanol and 0.1 g of 1-octanal or 1-heptanal as reaction medium, and 50 mg of 5 wt. % Pd/Al₂O₃ catalyst and 10 mg ethyl iodide as active catalytic system. The reactor was sealed and pressurized with 20 bar of H₂, heated up to 60° C. and kept under continuous stirring during at least 2 h (up to 15 h max). After reaction, the products were analyzed by GC and GC-MS. The preliminary catalytic results (alkyl aldehyde conversion and selectivity to the corresponding ethers) are presented in Table 1. Additional side products, such as acetals and hemiacetals, were also observed.

TABLE 1
Catalytic results on the etherification of alkyl aldehydes

| Substrate | | Time | Temperature | Conversion | Selectivity of | |
|---|---|---|---|---|---|---|
| Aldehydes | Alcohols | (h) | (° C.) | (%) | ether (%) | Structure |
| 1-Octanol | 1-Octanol | 16 | 60 | 96 | 95 | 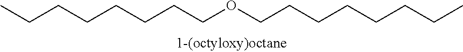 [SMILES: CCCCCCCCOCCCCCCCC] |
| 1-Heptanol | 1-Octanol | 15 | 60 | 94 | 96 | 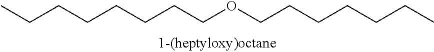 [SMILES: CCCCCCCCOCCCCCCC] |

1.2 Etherification of Aryl Aldehydes:

A 50 ml stainless still reactor was filled with 2 g of isopropanol or benzyl alcohol and 0.1 g of benzyl aldehyde as reaction medium, and 50 mg of 5 wt. % Pd/Al₂O₃ and 10 mg ethyl iodide as active catalytic system. The reactor was sealed and pressurized with 20 bar of H₂. The reactor was sealed and pressurized with 20 bar of H₂, heated up to 60˜80° C. and kept under continuous stirring during at least 2 h (up to 15 h max). After reaction the products were analyzed by GC and GC-MS. The catalytic results (aryl aldehyde conversion and selectivity to the corresponding ethers) are presented in Table 2. Additional side products, such as acetals and hemiacetals, were also observed.

TABLE 2
Catalytic results on the etherification of aryl aldehydes

| Substrate | | Time | Temperature | Conversion | Selectivity of | |
|---|---|---|---|---|---|---|
| Aldehydes | Alcohols | (h) | (° C.) | (%) | ether (%) | Structure |
| Benzyl aldehyde | Isopropanol | 3 | 60 | 35 | 65 | 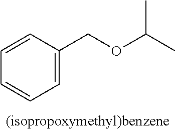 |
| Benzyl aldehyde | Benzyl alcohol | 2 | 80 | 40 | 51 | 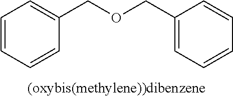 [SMILES: c1ccc(COCc2ccccc2)cc1] |

1.3 Etherification of Bio-Based Furfural and Vanillin:

A 50 ml stainless still reactor was filled with 2 g of alcohol (e.g. methanol, isopropanol, octanol, tert-amyl alcohol or furfuryl alcohol) and 0.1 g of furfural or vanillin, as it is specified in Table 3; then 50 mg of 5 wt. % Pd/Al₂O₃ catalyst and 10 mg ethyl iodide were added to the reaction medium. The reactor was sealed and pressurized with 20 bar of H₂, heated up to 60˜80° C. and kept under continuous stirring during at least 2 h (up to 15 h max). After reaction the products were analyzed by GC and GC-MS. The preliminary catalytic results (aryl aldehyde conversion and selectivity to the corresponding ethers) are presented in Table 3. Additional side products, such as acetals, hemiacetals and opening-ring derived products, were also observed.

TABLE 3
Catalytic results on the etherification of bio-based Furfural and Vanillin

| Substrate | | | Conversion | Selectivity of | |
|---|---|---|---|---|---|
| Aldehydes | Alcohols | Time (h) | (%) | ether (%) | Structure |
| Furfural | Methanol | 2 | 95 | 56 | 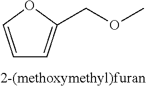 |
| Furfural | Isopropanol | 6 | 99 | 98 | 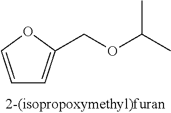 |
| Furfural | Tert- amyl alcohol | 15 | 15 | 91 | 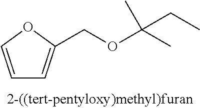 |
| Furfural | Octanol | 15 | 95 | 98 | 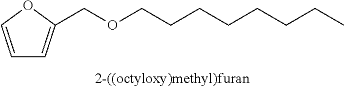 |
| Furfural | Furfuryl alcohol | 15 | 91 | 11 | 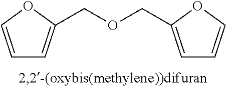 [SMILES: c1coc(COCc2ccco2)c1] |
| Vanillin | Isopropanol | 2 | 98 | 87 | 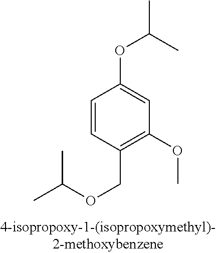 [SMILES: COc1cc(OC(C)C)ccc1COC(C)C] |

2. Catalyst Reusability

The catalyst after catalytic test has been reused for 5 times to check its stability. In a typical experiment, a 50 ml stainless steel reactor was filled with 2 g of isopropanol and 0.1 g of furfural as reaction medium, and 50 mg of 5 wt. % Pd/Al₂O₃ catalyst and 10 mg ethyl iodide as active catalytic system. The reactor was flushed with hydrogen for 5 times and pressured 20 bar H₂ as hydrogen source, then heated up to 60° C. and kept under continuous stirring for 2 h. After reaction, the catalyst was separated by centrifugation and the liquid phase was analyzed by GC. The separated catalyst was transferred to a clean autoclave without washing and drying, and then added fresh substrate (2 g isopropanol and 0.1 g furfural). The reactor was sealed and pressured with 20 bar H₂ and kept at 60° C. for 2 h under continuous stirring. The process was repeated for 5 times. The catalytic results (furfural conversion and selectivity to the corresponding ether) are presented in Table 4. Additional side products, such as acetals, hemiacetals and opening-ring derived products, were also observed.

TABLE 4
Reusability for 5 cycles.

| Cycles | Conversion (%) | Selectivity of ether (%) |
|---|---|---|
| 1 | 90 | 92 |
| 2 | 85 | 91 |
| 3 | 95 | 91 |
| 4 | 97 | 89 |
| 5 | 92 | 86 |

3. Pre-Treatment of Pd/Al₂O₃ with Iodide

Gas phase treatment by ethyl iodide: The initial Pd/Al₂O₃ catalyst was treated by chemical vapor deposition process with ethyl iodide as the vapor phase. Typical, put 200 mg Pd/Al₂O₃ in a 5 ml glass bottle, then 1 ml of ethyl iodide was added in a 50 ml stainless steel reactor and next set the bottle in it. The reactor was sealed and flushed with nitrogen for 3 times to remove the resident oxygen followed by heating the reactor at 60° C. for 2 h. To test the catalyst, in a 50 ml stainless steel reactor 2 g of isopropanol and 0.1 g of furfural were put, then 50 mg of iodide modified Pd/Al₂O₃ as a bifunctional catalyst were added for hydrogenation followed by etherification of furfural. The reactor was sealed and pressurized with 20 bar of H₂. The reaction was heated to 60° C. for 3 h under continuous stirring. After reaction the products were analyzed by GC and GC-MS. The preliminary conversion of furfural is 74% and the selectivity of corresponding ether is 91%. Additional side products, such as acetals, hemiacetals and opening-ring derived products, were also observed.

Liquid phase treatment by ethyl iodide: 200 mg of initial Pd/Al₂O₃ catalyst was added in a 50 ml stainless still reactor, then filled with 4 g of isopropanol and 20˜200 mg ethyl iodide. The reactor was sealed and pressurized with 20 bar of H₂, heated up to 60˜80° C. and kept under continuous stirring during at least 2 h. Then the catalyst was separated and washed with solvent for 5 times, followed by drying at vacuum oven at 80° C. overnight. To test the catalyst, in a 50 ml stainless steel reactor 2 g of isopropanol and 0.1 g of furfural were put, then 50 mg of iodide modified Pd/Al₂O₃ as a bifunctional catalyst were added for hydrogenation followed by etherification of furfural. The reactor was sealed and pressurized with 20 bar of H₂. The reaction was heated to 60° C. for 3 h under continuous stirring. After reaction the products were analyzed by GC and GC-MS. The preliminary conversion of furfural is 86% and the selectivity of corresponding ether is 92%. Additional side products, such as acetals, hemiacetals and opening-ring derived products, were also observed.

Liquid phase treatment by iodide: 200 mg of initial Pd/Al₂O₃ catalyst was mixed with 4 g of isopropanol and 10˜100 mg iodine in a glass bottle. Then the mixture was stirred continuously at room temperature for at least 2 h. Finally, the catalyst was separated and washed with solvent for 5 times, followed by drying at vacuum oven at 80° C. overnight. To test the catalyst, in a 50 ml stainless steel reactor 2 g of isopropanol and 0.1 g of furfural were put, then 50 mg of iodide modified Pd/Al₂O₃ as a bifunctional catalyst were added for hydrogenation followed by etherification of furfural. The reactor was sealed and pressurized with 20 bar of H₂. The reaction was heated to 60° C. for 3 h under continuous stirring. After reaction the products were analyzed by GC and GC-MS. The preliminary conversion of furfural is 89% and the selectivity of corresponding ether is 94%. Additional side products, such as acetals, hemiacetals and opening-ring derived products, were also observed.

4. Other Sources of I

In a typical experiment, a 50 ml stainless steel reactor was filled with 2 g of isopropanol and 0.1 g of furfural, then 50 mg of 5 wt. % Pd/Al₂O₃ catalyst and 18 mg HI (53% of water solution) or 15 mg NaI or 10 mg I₂ were added to the reaction medium. The reactor was sealed and pressurized with 20 bar of H₂, heated up to 60° C. and kept under continuous stirring for 2 h. After reaction the products were analyzed by GC and GC-MS. The catalytic results (furfural conversion and selectivity to corresponding ether) are presented in Table 5. Additional side products, such as acetals, hemiacetals and opening-ring derived products, were also observed.

TABLE 5
Catalytic results from other I source

| Additives | Conversion (%) | Selectivity of ether (%) |
|---|---|---|
| HI (53%) | 30.4 | 72.3 |
| NaI | 26.6 | 0.2 |
| I₂ | 53 | 62 |

5. Other Types of Supports and Metals

In a typical experiment, a 50 ml stainless steel reactor was filled with 2 g of isopropanol and 0.1 g of furfural, then 50 mg of 5 wt. % Pd/C or 5 wt. % Ru/Al₂O₃ catalyst or 5 wt. % Pt/C and 10 mg ethyl iodide were added to the reaction medium. The reactor was sealed and pressurized with 20 bar of H₂, heated up to 60° C. and kept under continuous stirring for 3 h. After reaction the products were analyzed by GC and GC-MS. The preliminary catalytic results (furfural conversion and selectivity to the furfuryl alcohol, tetrahydrofurfuryl alcohol, and corresponding ethers) are presented in Table 6. Additional side products, such as acetals, hemiacetals and opening-ring derived products, were also observed.

TABLE 6
Catalytic results over different supports and metals

| | | Selectivity (%) | | |
|---|---|---|---|---|
| | | Furfuryl | Tetrahydrofurfuryl | |
| Additives | Conversion (%) | alcohol | alcohol | Ether |
| Pd/C | 95 | 11 | 2 | 85 |
| Pt/C | 77 | 92 | 3 | 0.4 |
| Ru/Al₂O₃ | 18 | 96 | 0.7 | 0.1 |

1. A process for the synthesis of an ether compound, comprising reacting
an aldehyde and/or a ketone with an alcohol,
in the presence of (i) a metal/support heterogeneous catalyst and an iodine source, or (ii) a metal-iodine catalyst,
in a reactor, whereby the ether compound is obtained.

2. The process according to claim 1, further comprising (i) providing a protected aldehyde and/or a protected ketone, and deprotecting the protected aldehyde and/or the protected ketone to obtain said aldehyde and/or said ketone prior to said reacting step.

3. The process according to claim 1, further comprising providing a protected alcohol, and deprotecting the protected alcohol to obtain said alcohol prior to said reacting step.

4. The process according to claim 1, wherein an aldehyde is reacted with an alcohol, and wherein the aldehyde is selected from the group consisting of 1-octanal, 1-heptanal, benzyl aldehyde, furfural and vanillin.

5. The process according to claim 1, wherein the alcohol is selected from the group consisting of octanol, isopropanol, benzyl alcohol, methanol, isopropanol, tert-amyl alcohol and furfuryl alcohol.

6. The process according to claim 1, wherein the iodine source is selected from the group consisting of inorganic iodides, alkyl iodides and I₂.

7. The process according to claim 1, wherein the metal/support heterogenic catalyst comprises a support and a catalytically active metal.

8. The process according to claim 7, wherein the catalytically active metal is selected from the group consisting of Pd, Pt and Ru.

9. The process according to claim 7, wherein the support is selected from the group consisting of Al₂O₃, SiO₂Ga₂O₃ and ZrO₂.

10. The process according to claim 1, wherein the metal/support heterogeneous catalyst is Pd/Al₂O₃, and wherein the iodine source is CH₃CH₂I.

11. The process according to claim 1, wherein the process is carried out at a temperature in the range from 50-90° C.

12. The process according to claim 1, wherein the reacting is performed at an H₂-pressure in the range from 10-30 bar.

13. The process according to claim 1, wherein the reacting is performed for a time period in the range from 1-20 h.

14. A catalytic system comprising a metal/support heterogeneous catalyst, and an iodine source, wherein the metal is selected from the group consisting of Ru, Rh, Ir, Pd and Pt, wherein the support is selected from the group consisting of Al₂O₃, ZrO₂, C, SiO₂ and Ga₂O₃, and wherein the iodine source is selected from the group consisting of organic iodides, HI and I₂, and/or said catalytic system is under an H₂-pressure in the range from 10-30 bar.

15. A process for the preparation of a catalytic system, wherein said process comprises contacting a metal/support heterogeneous catalyst and an iodine source selected from the group consisting of organic iodides, HI and I₂ under an H₂-pressure in the range from 10-30 bar.

16. The process according to claim 1, wherein the iodine source is selected from the group consisting of CH₃CH₂I, NaI, HI and I₂.
